(19) United States
(12) Patent Application Publication
(10) Pub. No.: US 2026/0262991 A1
(43) Pub. Date: Sep. 10, 2026

(54) ANXIETY-AVOIDING METHOD AND SYSTEM FOR THE RECORDING OF HEALTH-RELATED SYMPTOMS AND ACTIVITIES

(71) Applicant: KONINKLIJKE PHILIPS N.V., EINDHOVEN (NL)

(72) Inventors: Wilhelmus Johannes Joseph Stut, Eindhoven (NL); Andreas Ejupi, Eindhoven (NL)

(21) Appl. No.: 19/163,615

(22) PCT Filed: Mar. 19, 2024
(86) PCT No.: PCT/EP2024/057332

§ 371 Date: Sep. 9, 2025

(30) Foreign Application Priority Data
Mar. 20, 2023 (EP) 23162812.4

(51) Int. Cl. A61B 5/318; A61B 5/00; A61B 5/0205; A61B 5/11

(52) U.S. Cl. CPC A61B 5/318; A61B 5/0002; A61B 5/0205; A61B 5/1118; A61B 5/1126; A61B 5/6833; A61B 5/7475; A61B 2562/0219

(57) ABSTRACT

A system includes a monitoring device including at least one sensor, the monitoring device configured to be worn by an associated patient and the at least one sensor configured to measure physiological sensor data of the associated patient wearing the monitoring device. An electronic processor is integrated with or in wireless communication with the monitoring device. The electronic processor is programmed to analyze the physiological sensor data to detect a health-related event; and prompt the associated patient to provide information via a user interface based on the detected health-related event.

### BACKGROUND

Health-related unobtrusive sensing systems enable replacement of continued hospitalization with obtrusive vital signs sensor technologies, centered around the individual, to provide remote monitoring of the subject's general health condition. Vital signs monitoring typically includes monitoring one or more of the following physical parameters: heart rate (HR), blood pressure (BP), respiratory rate (RR), core body temperature and blood oxygenation (SpO₂).

Current ambulatory ECG monitors and mobile cardiac telemetry systems gather ECG data in ambulatory settings (e.g. in the patient's home environment or on-the-go). In such systems, a sensor is placed in a patch on the chest. The sensor communicates wirelessly with a dedicated smartphone which is connected via the Internet to the clinical service center. In other embodiments, the sensor may be directly connected to the Internet. Some examples of such devices include the ePatch (Extended Holter Monitor) and MCOT (Mobile Cardiac Telemetry) devices available from Philips ECG Solutions. These devices provide a wearable single-use electrodes patch onto which an electronics module attaches to form a patient-worn device.

In such systems, when patients feel a symptom, they can inform the sensor about the occurrence of a symptom by touching it in a specific way (e.g., double-tapping the sensor, a dedicate button on the sensor, and so forth). Touching the sensor in a specific way adds a mark to the ECG data stream. By touching the sensor, the sensor only registers that a symptom has occurred, but the patient has not described or recorded the symptoms and activity yet.

The description of the symptom and the activity is typically done via an app on a smartphone of the patient. When patients feel a symptom, they can select the symptom recording function of the app, and provide details like the nature of the symptom (e.g. fainting, dizziness, chest pain, and so forth), the activity during which the symptom occurred (e.g. walking, cycling), and/or the (perceived) intensity of the activity (e.g. light, medium, heavy, etc.).

The drawback of traditional patient symptom records is that they are often inaccurate or incomplete. When patients experience a symptom, they may simply forget to describe the activity and symptom, or even ignore it. Furthermore, so-called “silent” cardiac events cannot be noticed since they (by definition) involve no symptoms at all, which means that the patient even does not know s/he should describe the activity.

In addition, when the ECG system detects a cardiac event, it could ask the patient to record their activity and symptom. However, this approach may lead to patient anxiety and uncertainty, especially when the event is a silent cardiac event.

The following discloses certain improvements to overcome these problems and others.

### SUMMARY

In one aspect, a system includes a monitoring device including at least one sensor, the monitoring device configured to be worn by an associated patient and the at least one sensor configured to measure physiological sensor data of the associated patient wearing the monitoring device. An electronic processor is integrated with or in wireless communication with the monitoring device. The electronic processor is programmed to analyze the physiological sensor data to detect a health-related event; and prompt the associated patient to provide information via a user interface based on the detected health-related event.

In another aspect, a method of monitoring an associated patient includes analyzing physiological sensor data of a patient obtained by a monitoring device to detect a health-related event; and prompting the associated patient to provide information via an associated mobile device based on the detected health-related event.

One advantage resides in providing a reliable remote monitoring system for monitoring a patient.

Another advantage resides in ensuring accurate patient symptoms records.

Another advantage resides in prompting patients to describe their symptoms based on collected physiological data of the patient.

Another advantage resides in prompting patients at random times to describe their symptoms.

Another advantage resides in reducing patient anxiety and uncertainty in reporting their symptoms.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows a patient monitoring system in accordance with the present disclosure.

FIG. 2 shows an example flow chart of operations suitably performed by the system of FIG. 1.

FIG. 3 shows an example of a process performed by the system of FIG. 1.

FIG. 4 shows an example of data collected by the system of FIG. 1.

### DETAILED DESCRIPTION

As used herein, the singular form of “a,” “an”, and “the” include plural references unless the context clearly dictates otherwise. As used herein, statements that two or more parts or components are “coupled,” “connected,” or “engaged” shall mean that the parts are joined, operate, or co-act together either directly or indirectly, i.e., through one or more intermediate parts or components, so long as a link occurs. Directional phrases used herein, such as, for example and without limitation, top, bottom, left, right, upper, lower, front, back, and derivatives thereof, relate to the orientation of the elements shown in the drawings and are not limiting upon the scope of the claimed invention unless expressly recited therein. The word “comprising” or “including” does not exclude the presence of elements or steps other than those described herein and/or listed in a claim. In a device comprised of several means, several of these means may be embodied by one and the same item of hardware.

The disclosed system aims to avoid anxiety by not only asking patients about their symptoms and activity when the system has detected a cardiac event, but also at additional moments. When the patient starts using the disclosed system, the patient will be explained that the disclosed system can ask him/her to describe symptoms and activities after a cardiac event, but also at other moments. Hence, since a request to describe symptoms and activities does not imply that the patient just had a cardiac event, this approach is assumed to reduce (or even prevent) patient anxiety and uncertainty.

The moments at which the system sends these recording requests, depend amongst others on the detected cardiac events, time of the day, and previous spontaneous recording behavior of patients. Furthermore, when the disclosed system has an accelerometer to detect the activity level over time, it may ask patients to provide symptom and activity information based on the observed activity level or activity pattern, possibly in combination with previous spontaneous recording behavior of patients. In both cases the system learns from previous spontaneous recording behavior of patients and triggers new reporting requests based on similar characteristics.

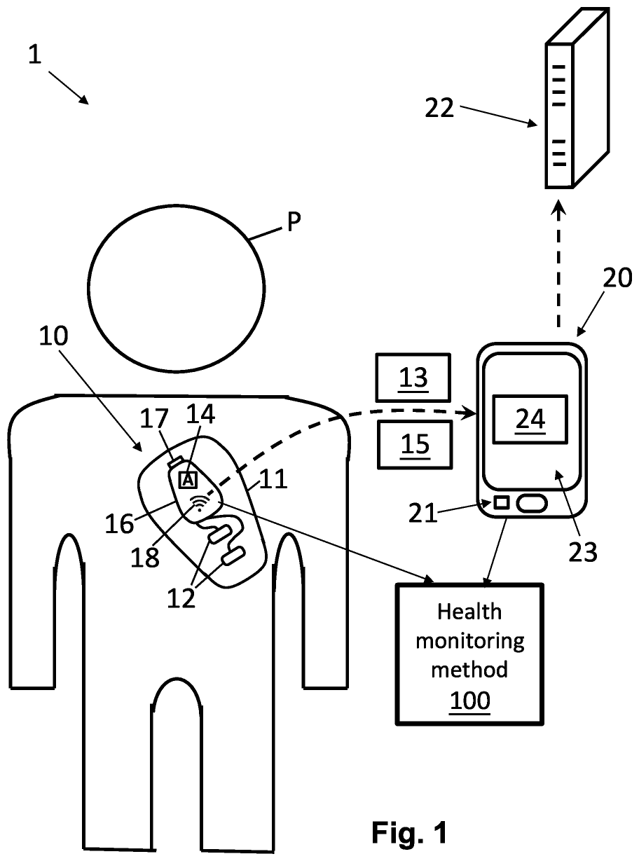

With reference to FIG. 1, a system 1 for monitoring an associated patient P is shown. As used herein, the term “patient” (and variants thereof) refers to, and includes, an outpatient, a discharged patient, a patient undergoing screening using the monitoring device as part of an annual medical checkup, or other person whose health condition is to be monitored. As shown in FIG. 1, the system 1 includes a wearable monitoring device 10 that is wearable by, or otherwise attached to, the patient P. The wearable monitoring device 10 can include any suitable monitoring device, such as a Mobile Cardiac Outpatient Telemetry (MCOT)® device (available from Philips ECG Solutions, Malvern, Pennsylvania, USA), or a medical wearable device, a torso-worn vital signs health patch, a wrist-worn watch, a chest strap, a smart garment, medical ear buds/over the ear, a forehead or nose sensor, a smart ring, or so forth.

The illustrative wearable monitoring device 10 includes a single-use electrodes patch 11 with an ECG sensor 12 (e.g., embodied as electrodes 12) for collecting cardiac data 13, and an accelerometer 14 collecting accelerometer data 15. The illustrative monitoring device 10 includes an electronics module 16 that attaches onto the electrodes patch 11, and the accelerometer 14 may be integrated into the electronics module 16. The electronics module 16 includes at least one microprocessor or microchip (not shown) configured (e.g. programmed) to optionally preprocess the ECG signals from the ECG sensor (electrodes) 12 to produce the cardiac data 13 and/or optionally preprocess the data from the accelerometer 14 to produce the accelerometer data 15. The device 10 including the electrodes patch 11 and the attached electronics module 16 is adhesively secured to the chest or other anatomy of the patient P after suitable preparation (e.g. cleaning and/or shaving) of the skin. The use of the separate electrodes patch 11 and electronics module 16 advantageously enables the single-use electrodes patch 11 to be a low-cost component that can be replaced as needed over the course of a patient monitoring session (which may extend over multiple days or weeks) while re-using the more expensive electronics module 16. However, other arrangements are contemplated such as having the patch 11 and electronics 16 constructed as a unitary single-use unit. Although not shown, it will be appreciated that the wearable monitoring device 10 also includes an on-board battery or other on-board electrical power source to power the electronics module 16. The on-board battery may, for example, be integrated with the electronics module 16, which may include a recharging port connector 17 for recharging the electronics module 16, or the electronics module 16 could be placed on a wireless inductive recharging station to recharge it if needed during a patient monitoring study.

The wearable monitoring device 10 more generally can include one or more sensors configured to measure physiological sensor data of the patient P wearing the monitoring device 10. The measured physiological sensor data of the patient P is used to detect a health-related event of the patient P. As shown in FIG. 1, the monitoring device 10 includes two sensors—an electrocardiogram (ECG) sensor 12 and an accelerometer 14. The ECG sensor 12 is configured (e.g., comprising skin-contacting electrodes with silver/silver chloride coatings, for example) to measure cardiac data 13 of the patient P, and the health-related event comprises a cardiac event detected by analysis of the cardiac data 13. In addition, the ECG sensor 12 can be replaced with (or supplemented by) any other suitable sensor to measure a corresponding vital sign of the patient (e.g., SpO₂, electroencephalogram (EEG), and so forth). The accelerometer 14 is configured to measure patient activity data of the patient P, more specifically, accelerometer data 15.

The wearable monitoring device 10 also includes a wireless transmitter or transceiver 18 (referred to hereinafter as a transceiver 18), which may optionally be integrated with the electronics module 16. The transceiver 18 is integrated with or in wireless communication with the monitoring device 10 to transmit the patient data (e.g. the cardiac data 13 and accelerometer data 15) to a mobile device 20. In some embodiments, the electronic processor 16 is configured to collect and optionally preprocess the cardiac data 13 from the ECG sensor 12 and/or the accelerometer data 15 from the accelerometer 14, and the transceiver 18 is configured to transfer the cardiac data 13 and/or the accelerometer data 15 to the mobile device 20 (e.g., a cellphone or other smart device, or a dedicated medical monitoring device) operable by the patient P. In a typical arrangement, the transceiver 18 is a low-power wireless transceiver (e.g., Bluetooth™, Zigbee™, or the like) that connects with low power to the mobile device 20, thus placing a low power draw on the on-board battery of the wireless monitoring device 10. The patient P can be prompted to provide information, including at least health-related symptoms that the patient P is experiencing, using the mobile device 20 running an application (“app”) executed by an electronic processor 21 of the mobile device 20. In some examples, this information can be transmitted to a clinical health information system 22 comprising a server computer, for example transmitted over the Internet via a 3G/4G/5G wireless cellular network, Wi-Fi, various combinations thereof, and/or another wireless communication protocol. Using the intermediary mobile device 20 has certain advantages such as providing a display 23 on which a user interface (UI) 24 can be displayed, e.g. to present a diary for the patient to record health symptoms. The mobile device 20 can also perform some or all processing of the cardiac and accelerometer data 13 and 15 instead of performing that processing at the on-board electronics module 16 of the wearable monitoring device 10. However, it is alternatively contemplated to omit the separate mobile device 20 and instead have all processing performed by the electronics module 16 of the wearable monitoring device 10, and to have the transceiver 18 of the wearable monitoring device 10 wirelessly communicate directly with the clinical health information system 22. In such embodiments the wearable monitoring device may also include a display for presenting the UI 24—for example, the wearable monitoring device 10 could have the form factor of a wristwatch.

The processing of the patient data 13 and 15 can be variously distributed between the electronic module 16 of the wearable monitoring device 10 and the mobile device 20. Hence, an electronic processor 16, 21 is referred to herein, to indicate the combined processing capacity of the electronic module 16 of the wearable monitoring device 10 and the mobile device 20. In general, processing described as being performed by the electronic processor 16, 21 may be performed entirely by the electronic module or processor 16 of the wearable monitoring device 10, or entirely by the electronic processor 21 of the mobile device 20, or the processing may be shared between them.

As further diagrammatically indicated in FIG. 1, the electronic processor 16, 21 can be configured to perform a health status monitoring method 100 of monitoring the patient P by analyzing the cardiac data 13 and/or the accelerometer data 15.

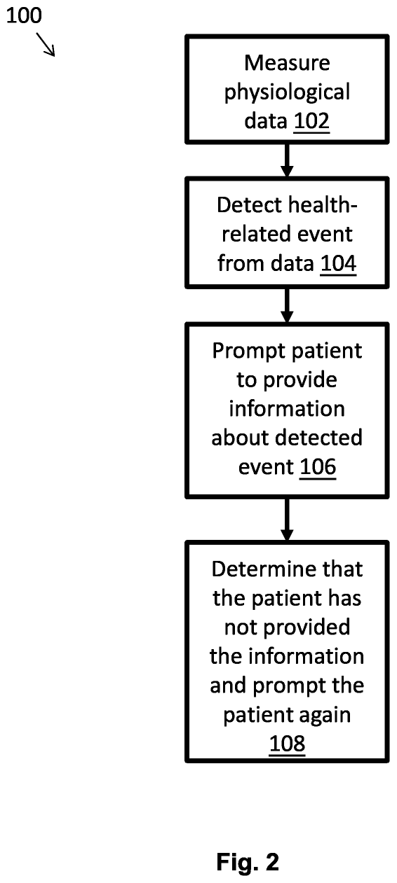

With reference now to FIG. 2, an illustrative embodiment of the health status monitoring method 100 is shown by way of a flowchart. To begin the method 100, the monitoring device 10 is attached to the patient P. At an operation 102, the cardiac data 13 is measured by the ECG sensor 12 and the accelerometer data 15 is measured by the accelerometer 14. At an operation 104, the electronic processor 16, 21 is configured to analyze the cardiac data 13 to detect a health-related event of the patient P (and the accelerometer data 15 to detect an activity-related event of the patient P). At an operation 106, the electronic processor 16, 21 is programmed to prompt the patient P to provide information via the mobile device 20 based on the detected health-related event. The patient P providing the information is also sometimes referred to herein as making a diary entry. To do so, the electronic processor 16, 21 is configured to control the mobile device 20 to display the user interface (UI) 24 on a screen of the mobile device 20. The UI 24 is configured to receive inputs from the patient P to input the information. In some embodiments, the UI 24 may be a graphical user interface (GUI). In some embodiments, the UI 24 provides the prompt by presenting a diary entry UI comprising a set of user dialogs with symptoms listed (e.g., fainted, dizzy, chest pain, light headed, skipped heartbeat, shortness of breath, heart racking, or so forth) which can be selected by the patient by checking associated boxes or the like, along with user-selectable listed activities (resting, light activity, medium activity, heavy activity); and/or may provide a freeform text entry dialog for the user to describe his or her symptoms in greater detail. Upon completion of the diary entry it is suitably transmitted from the mobile device 20 to the clinical health information system 22.

In general, the patient can initiate a diary entry manually, for example by selecting a “Make diary entry” button or the like shown on the UI 24. For example, the patient's physician or the supplier of the system 1 will instruct the patient to initiate a diary entry any time the patient experiences a symptom. This provides valuable contextual information for consideration by the patient's physician when reviewing the patient data collected by the patient monitoring session.

However, analysis of the cardiac data 13 by the electronic processor 16, 21 can detect certain types of cardiac events that may not be perceived by the patient. These are sometimes referred to as silent cardiac events. More generally, physiological sensor data from the monitoring device 10 can be analyzed by the electronic processor 16, 21 to detect a health-related event that might not be perceived by the patient. As another example, an EEG monitor may detect abnormal brain activity that is not consciously recognized by the patient. Hence, reliance on manually initiated diary entries will not be effective to obtain contextual information for such silent health symptoms. Moreover, even if the patient feels a symptom he or she may fail to proactively initiate a diary entry in a timely fashion to provide the contextual information. The operation 104 of the health monitoring method 100 advantageously detects silent health-related events or health-related events that are ignored by the patient, and automatically initiates the prompt 106.

The operations 104 and 106 can be performed in a variety of manners. In particular, the electronic processor 16, 21 is programmed to additionally prompt the patient P to provide the information via the mobile device 20 at times not associated with a detected cardiac event. In one example, the electronic processor 16, 21 is configured to prompt the patient P to provide the information via the mobile device 20 at a random time independent of whether a health-related event was detected. As used herein, the term “random” can comprise a completely random or a pseudorandom occurrence that can be deterministic, but not as regular intervals. Advantageously, this approach compensates for that fact cardiac events are often silent in combination with the problem of making the patient anxious about reporting their symptoms. In other words, if the patient P knows or suspects that the system 1 prompted a diary entry due to detection of a silent cardiac event, the very fact of the prompt can induce anxiety in the patient P. By additionally making random prompts that are unrelated to detected silent cardiac events, the patient P comes to expect prompts to occur on occasion, and can be accurately informed that the diary entry prompts are not usually related to silent cardiac events. To do so, the electronic processor 16, 21 is configured to detect, from measured activity data 15 of the patient P, a sleeping time period during which the patient P is sleeping, and not prompt the patient P to provide the information during the determined sleeping time period. In another example, the electronic processor 16, 21 is configured to detect, from measured activity data 15 of the patient P, a period of activity (e.g., running, walking, exercising, etc.) and/or inactivity (e.g., laying down, watching television, etc.), and at the end of the activity session (or during the inactivity session), prompt the patient P to provide the information via the mobile device 20. These are merely examples, and should not be construed as limiting.

At an operation 108, the electronic processor 16, 21 is programmed to determine whether the patient P has provided information via the mobile device 20 describing the detected health-related event, and the prompt to provide information based on the detected health-related event is further based on it being determined that the patient P has not provided the information.

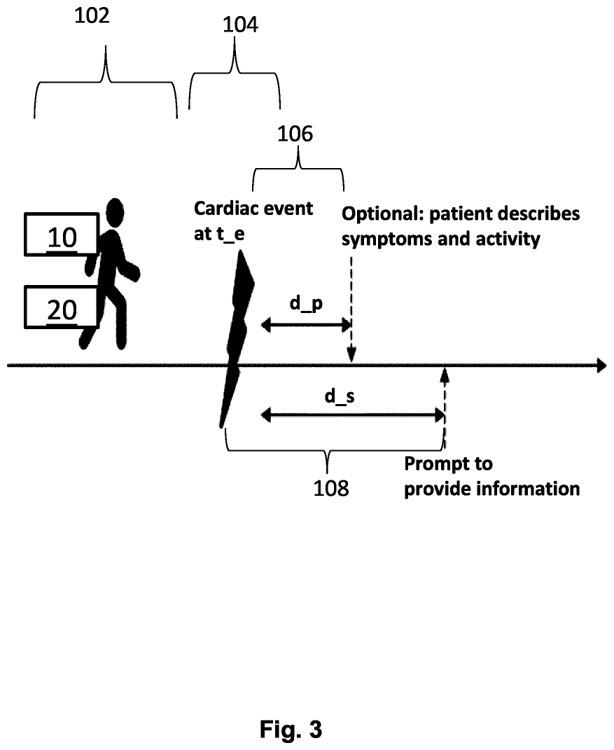

FIG. 3 shows the operations of the method 100 as a timeline. The operation 102 is shown with the patient P wearing the monitoring device 10 and having the mobile device 20 on their person, and the physiological data 13, 15 is collected. Based on the physiological data 13, 15, the monitoring device 10 detects that at time t_e (time of event) a cardiac event has occurred (i.e., the operation 104). At the operation 106, after a delay of d_p (i.e., a delay by the patient P) the patient P describes the symptom and activity on their own via the mobile device 20. At the operation 108, after a delay of d_s (i.e., a delay by the system 1) the monitoring device 10 checks whether the patient P has described the health-related event. If not, at a time t_e+d_s, the monitoring device 10 asks the patient P to describe the symptom and activity around t_e by inputting the information to the mobile device 20. The value of d_s can be fixed (e.g. 5 minutes), but can also depend on the spontaneous recording behavior of the patient, i.e. the observed d_p values of the patient P. Example values for d_s are, for example, d_s=average (observed d_p values)+2 minutes, d_s=average (observed d_p values)*2, and d_s=average (observed d_p values)+standard deviation (observed d_p values). The system 1 may also let the value of d_s depend on the observed d_p values in other patients. Especially in the first hours or days of the monitoring period, when the patient P had no cardiac events yet (and therefore no d_p values), this is an attractive approach.

To prevent patient anxiety, the system 1 not only asks patients about their symptoms and associated activity when it has detected a cardiac event, but also at so-called additional moments. Example additional moments are, for example, randomly selected moments (e.g. random moments between 8 a.m. and 10 p.m.), fixed moments (e.g. 8 a.m., 2 p.m., and 10 p.m.), a random delay after the last symptom and activity recording (e.g. 4 to 6 hours later), a moment in time similar to previous moments where symptoms were reported (e.g. before lunch or bed time), and so forth. The system 1 should not do this when the patient P has just reported symptoms and activity, or when the system 1 has just detected a cardiac event at t_e and is still waiting until t_e+d_s. In that case, a next additional moment will be determined.

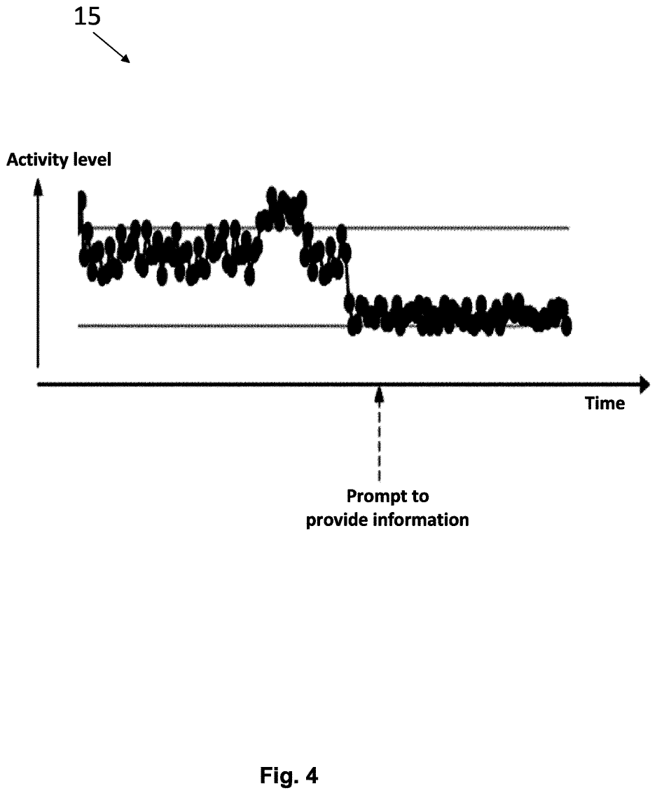

FIG. 4 shows an example of the accelerometer data 15 being used to detect the activity-related events of the patient P. The accelerometer data 15 can be used to detect activity sessions (e.g. walking or running sessions). When an activity session has ended, the system 1 can ask the patient P whether they had symptoms during this activity session, and what type of activity (e.g. walking, running, cycling) they were doing, as shown in FIG. 4. In some examples, the activity type information can also be used to train a machine learning algorithm for automated activity type detection.

The accelerometer 15 can also be used to detect periods of inactivity (e.g. the patient has been sitting for a longer period) where it is more likely that a patient P has time to report and reacts calmly to a reporting request. Furthermore, information from the mobile device 20 (e.g. a phone in use) and/or other sensors (e.g. low heart rate from ECG signal) can be used/added to determine these periods of inactivity. Some examples can include, for example, a low heart rate from ECG signal, a low heart rate from ECG signal AND phone not in use, and so forth. The accelerometer 14 can also be used to detect activity patterns preceding spontaneous reporting. For example, the system 1 may notice that a patient P always spontaneously reports symptoms after a night of disturbed sleep. If such an activity pattern occurs again, but the patient P has not spontaneously reported symptoms and activity afterwards, the system 1 may ask the patient P to report symptoms and activity.

When asking the patient P about their symptoms and associated activity (i.e., after a cardiac event or at an additional moment), the system 1 should first ask whether the patient P experienced symptoms at a particular moment or during a particular period. Only when the patient P confirms, the UI 24 to describe the symptoms will be shown. The UI 24 to describe the activity are always shown. The request to describe symptoms and activity may refer to the present (i.e. the current moment or the past n minutes), or to the past (i.e. a moment or a period in the past). When the system 1 asks the patient P to describe their symptoms and activity, it may send a notification (e.g. a push notification on the mobile device 20) to draw their attention.

In some examples, the patient P may be sleeping when the system 1 asks to record symptoms and activity. To prevent that the system 1 disturbs the sleeping patient P, the system 1 can be configured to not send puh notifications when the patient P is sleeping (e.g. detected via the accelerometer data 15) or during configurable time zones (e.g. 10:00 PM to 08:00 AM). The system 1 can be configured to not present the UI 24 when the patient P is sleeping or during configurable time zones (e.g. 10:00 PM to 08:00 AM). In the latter case, the accelerometer 14 can be used to determine when the patient P got out of bed in the morning. After a configurable delay (e.g. 15 minutes after getting out of bed), the system 1 may ask whether the patient P felt symptoms, for example, during the past night (i.e., without indicating a specific moment) or at specific moments during the night (e.g. at 03:40 a.m.). These moments may correspond to detected cardiac events, or be additional moments.

The system 1 may also ask the patient to take additional measurements (e.g. with an SpO2 sensor) after a cardiac event or at additional moments. In another example, a number of requests to report symptoms and activity may be bound to a (i.e., daily) limit. Different limits may apply to the number of requests after cardiac events, the number of additional moments, or the sum thereof.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A system comprising:
a monitoring device configured to be worn by an associated patient, including a physiological sensor, configured to measure physiological sensor data of the associated patient wearing the monitoring device; and
an accelerometer configured to measure activity data of the associated patient;
an electronic processor integrated with or in wireless communication with the monitoring device, the electronic processor programmed to:
analyze the physiological sensor data to detect a health-related event; and
detect, from the measured activity data, a period of inactivity of the associated patient; and
prompt the associated patient to provide information via a user interface based on the detected health-related event when a period of inactivity has been detected.

2. The system of claim 1, wherein the electronic processor is further programmed to:
prompt the patient to provide the information via the user interface at a random time independent of whether a health-related event was detected.

3. The system of claim 2, wherein the electronic processor is further programmed to:
detect, from measured activity data of the associated patient, a sleeping time period during which the associated patient is sleeping;
wherein the electronic processor is programmed to not prompt the associated patient to provide the information during the sleeping time period.

4. The system of claim 2, wherein the electronic processor is further programmed to additionally prompt the associated patient to provide the information via the user interface at times not associated with a detected health-related event.

5. The system of claim 1, wherein the physiological sensor comprises an electrocardiogram (ECG) sensor configured to measure cardiac data of the associated patient, and the health-related event comprises a cardiac event detected by analysis of the cardiac data.

6. The system of claim 1, wherein the electronic processor is integrated with the monitoring device, and the monitoring device further includes a wireless transmitter or transceiver and is disposed on a patch attachable to the associated patient.

7. (canceled)

8. (canceled)

9. The system of claim 1, wherein the electronic processor is programmed to:
determine whether the associated patient has provided information describing the detected health-related event; and
wherein the prompt to provide information based on the detected health-related event is further based on it being determined that the associated patient has not provided the information.

10. (canceled)

11. (canceled)

12. The system of claim 1, wherein the information includes at least health-related symptoms that the associated patient is experiencing.

13. (canceled)

14. A method of monitoring an associated patient, the method comprising:
analyzing physiological sensor data of a patient obtained by a physiological sensor to detect a health-related event;
detecting, from activity data measured by an accelerometer, a period of inactivity of the associated patient; and
prompting the associated patient to provide information via an associated mobile device based on the detected health-related event when a period of inactivity has been detected.

15. (canceled)
